(19) United States
(12) Patent Application Publication
(10) Pub. No.: US 2001/0046473 A1
(43) Pub. Date: Nov. 29, 2001

(54) Gastric-retained pharmaceutical composition and method for its use

(76) Inventor: Jerome Besse, Listrac Medoc (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/886,991

(22) Filed: Jun. 25, 2001

Related U.S. Application Data
(63) Continuation of application No. 09/403,170, filed on Oct. 18, 1999, now abandoned.
Application No. 09/403,170 is a 371 of international application No. PCT/FR98/00755, filed on Apr. 15, 1998.

(30) Foreign Application Priority Data
Apr. 18, 1997 (FR) 9704803

(51) Int. Cl. A61K 9/20; A61K 31/165

(52) U.S. Cl. 424/44; 514/617

(57) ABSTRACT

Pharmaceutical composition for gastric residence, characterized in that it comprises:

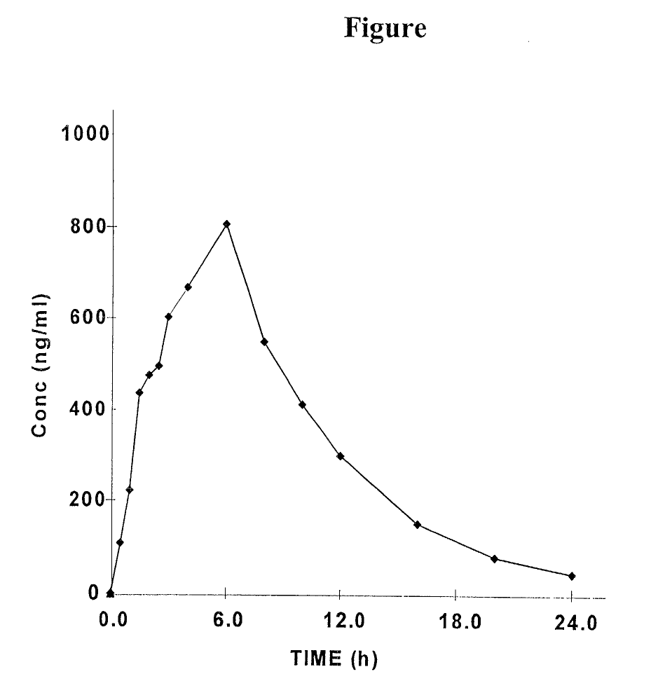

(a) an active principle consisting of a benzamide or a benzamide salt,

(b) a carbon dioxide generator system, and

(c) a means allowing the partial retention of the carbon dioxide generated by the said carbon dioxide generator system.

The present invention relates to pharmaceutical compositions for gastric residence, comprising an active principle from the benzamides family.

The benzamides are chemical compounds whose structure comprises the following motif:

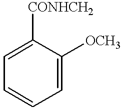

Certain benzamides are useful as active principle of medicaments intended for the treatment, especially, of disorders of the central nervous system. Such benzamides can consist of amisulpride, tiapride, sulpiride, their salts, if necessary, their enantiomers and the salts of these enantiomers, as well as some of their derivatives.

These benzamides can be administered by the oral route. However, the Applicant has been able to verify that administration by the oral route of these benzamides can lead to a low and/or irregular bioavailability. The term “bioavailability” is understood here as meaning the fraction of active principle which is absorbed from its pharmaceutical form and which reaches the plasma.

A low or irregular bioavailability can be the result of several factors among which it is possible to mention: a low solubility or a very slow dissolution of the active principle or of the pharmaceutical form which contains it; instability of the active principle, either over the entire length of the gastrointestinal tract, or in one part of it only; enzymatic degradation in the mucous membrane or at the hepatic level of the active principle; slow or incomplete absorption of the active principle owing to a slow passive diffusion through the intestine, or, in the case of an active mechanism, a saturation of the transport system.

It is known that the bioavailability of certain active principles can be modified by means of a prolonged release formulation which releases the active principle over the entire length of the gastrointestinal tract.

The Applicant, however, has been able to establish that such a formulation is not suitable for compounds of the benzamides family. In fact, the Applicant has been able to determine that the benzamides are generally poorly absorbed at the colonic level in man, but that, on the other hand, they are better absorbed in the small intestine. For certain of these benzamides, absorption takes place quasi-exclusively in the upper parts of the small intestine, that is to say the jejunum, the duodenum or the proximal ileum.

Continuing its research, the Applicant then considered improving the bioavailability of the benzamides by formulating them in the form of a pharmaceutical composition for gastric residence favouring an absorption at the level of the small intestine, or even, more specifically, the upper parts of the small intestine.

The invention thus consists of a pharmaceutical composition for gastric residence, characterized in that it comprises:

(a) an active principle consisting of a benzamide or a benzamide salt,

(b) a carbon dioxide generator system, and

(c) a means allowing the partial retention of the carbon dioxide generated by the said carbon dioxide generator system.

The figure represents the profile of plasma release, in man, of tiapride hydrochloride, this profile being obtained with a pharmaceutical composition according to the invention.

When the active principle is a compound comprising one or more centres of asymmetry, the term “benzamide” in the sense of the present invention, and unless stated otherwise, covers the different enantiomers or diastereoisomers of these compounds, including their mixtures, in particular their racemic mixtures.

A pharmaceutical composition for gastric residence is intended to reside for more than one hour in the stomach with a view to prolonged and/or controlled release of the active principle.

A pharmaceutical composition for gastric residence according to the invention has the advantage of being able to float on the surface of liquids contained in the stomach, and of being able to do this very rapidly after having been absorbed. It has thus been possible to verify that a composition according to the invention is able to float less than two minutes after having been contacted with an aqueous liquid.

It is very important that the flotation occurs as rapidly as possible after absorption in order to avoid the pharmaceutical composition being expelled from the stomach. In fact, it is generally considered that if the pharmaceutical composition for gastric residence does not float within the three minutes following absorption in a fasting subject, the probability that it is evacuated from the stomach becomes unacceptable.

The invention relates more particularly to active principles consisting of sulpiride, amisulpride, their salts, their enantiomers and the salts of these enantiomers, as well as to tiapride, its oxide and its salts.

Sulpiride or 5-(aminosulphonyl)-N-[(1-ethyl-2-pyrrolidinyl)methyl]-2-methoxybenzamide and its preparation process are described in the special French Medicine Patent No. 4879M, the teaching of which is incorporated in full in the present description. Sulpiride is a useful neuroleptic in the treatment of acute and chronic psychoses (i) for disinhibitory dosage: psychoses or predominant withdrawal states, apragmatism, abulia or (ii) for antiproductive dosage: delirious or confusional psychoses, schizophrenia.

Tiapride or N-[2-(diethylamino)ethyl]-2-methoxy-5-(methylsulphonyl)benzamide, its salts, its oxide and processes for their preparation are described in French Patent No. 75 09808, the teaching of which is incorporated in full in the present description. Tiapride, in particular in hydrochloride form, is a useful neuroleptic in the treatment of restlessness and aggressivity of insane subjects, behavioural disorders when they are manifested by hyperactivity, aggressivity or irritability phenomena, especially in alcoholics and the old, motor behavioural disorders such as, for example, tremors, spontaneous or latrogenic neuromuscular dyskinesias, abnormal movements such as chorea, tics, hemiballism, sensitive behavioural disorders such as, for example, cephalalgias, migraines, various pains, especially intense and stubborn pains. The present invention is more particularly suited to tiapride hydrochloride.

Amisulpride or 4-amino-N-[(1-ethyl-2-pyrrolidinyl)methyl]5-(ethylsulphonyl)-2-methoxybenzamide, its enantiomers and some of its derivatives are described in French Patent No. 78 01632, the teaching of which is incorporated in full in the present description. The invention is very particularly suitable for amisulpride per se, that is to say 4-amino-N-[(1-ethylpyrrolidin-2-yl)methyl]-5-(ethylsulphonyl)-2-methoxybenzamide, its laevorotatory ((S)-(−)-amisulpride) and dextrorotatory ((R)-(+)-amisulpride) enantiomers, mixtures of these enantiomers, the tartrates of amisulpride per se and of its enantiomers, as well as mixtures of these tartrates. A preferred tartrate consists of the compound described in Example IV of the Patent FR 78 01632, that is to say the (D) tartrate of (S)-(−)-amisulpride, in other words the [S-(R*,R*)]-2,3-dihydroxybutanedioate of (S)-(−)-4-amino-N-[(1-ethylpyrrolidin-2-yl)methyl]-5-(ethylsulphonyl)-2-methoxybenzamide. Amisulpride is a neuroleptic used in the treatment of psychoses, more particularly in the treatment of paranoid and productive schizophrenias, of acute delirious psychoses, as well as in the treatment of deficient schizophrenic states, residual psychotic developments and inhibition states with slowing. Amisulpride is also useful in the treatment of dysthymia.

Besides the benzamides mentioned above, other benzamides can be employed within the context of the present invention, such as metoclopramide, veralipride, alizapride or clebopride.

The carbon dioxide generator system has the principal function of forming carbon dioxide in the form of bubbles. These bubbles contribute to rapidly introducing, and then to maintaining, the pharmaceutical composition of the invention at the surface of the liquids contained in the stomach.

A suitable carbon dioxide generator system in a pharmaceutical composition according to the invention generally comprises at least one carbon dioxide generator agent. The carbon dioxide generator agent is usually a carbonate of an alkali or alkaline earth metal, such as calcium carbonate, or a bicarbonate of an alkali metal, preferably sodium bicarbonate.

Such a carbon dioxide generator system, formed solely of a carbon dioxide generator agent, only commences to form bubbles of carbon dioxide after having been contacted with a medium of acidic pH, generally that of the stomach.

In order to accelerate the formation of the bubbles of carbon dioxide, and thus to improve the flotation of the pharmaceutical composition for gastric residence of the invention, it is preferred to employ a carbon dioxide generator system which is independent of the pH. Such a system can comprise a carbon dioxide generator agent such as those mentioned further above, as well as at least one acidic compound chosen from the group formed by monocarboxylic acids such as lactic acid, polycarboxylic acids and partial salts of polycarboxylic acids. As acidic compounds, it is more particularly possible to mention tartaric, maleic, malonic, malic, fumaric, succinic, adipic and citric acids and their partial salts, such as monosodium citrate.

In such a carbon dioxide generator system, the content of acidic compound is generally chosen such that the number of moles in the said acidic compound with respect to the number of moles in the said carbon dioxide generator agent is between 0.7 and 1.4 times the stoichiometric amount. However, if the active principle or any other component entering into the formulation of the composition according to the invention has a basic character, it can be necessary as a consequence to increase the content of acidic compound.

The means allowing the partial retention of the carbon dioxide generated by the carbon dioxide generator system must allow diffusion of the carbon dioxide in a controlled manner. It must prevent the too-rapid diffusion of carbon dioxide which would lead to a too-short flotation in time of the pharmaceutical composition according to the present invention. Conversely, it must allow a sufficient diffusion of the carbon dioxide to ensure a determined flotation time of the said composition in the stomach, as well as the sufficient diffusion of water or of an aqueous compound in the composition according to the invention.

This means can consist of a porous mineral matrix, especially of matrices based on silicates or calcium fluoride, or, preferably, of a polymer. According to a particularly advantageous aspect of the invention, the said polymer consists of at least one hydrophilic polymer.

The hydrophilic polymers suitable for a pharmaceutical composition according to the invention are those able to form a hydrocolloid gel in contact with an aqueous liquid, in particular aqueous liquids contained in the stomach. By way of such hydrophilic polymers, it is possible to mention (i) the natural polysaccharides such as alginates, xanthan gum, guar gum or carob gum, (ii) the semisynthetic polysaccharides, in particular cellulose derivatives such as methylcellulose, ethylcellulose, methylhydroxyethylcellulose, carboxymethylcellulose and its salts such as sodium carboxymethylcellulose or calcium carboxymethylcellulose and, preferentially, hydroxypropylcellulose, hydroxypropylmethylcellulose and mixtures of hydroxypropylcellulose and hydroxypropylmethylcellulose or (iii) vinyl polymers, synthetic hydrophilic polymers such as polymers derived from acrylic and methacrylic acids and their salts, such as polyacrylates, especially those marketed under the trade name Carbopol®, amino acid polymers such as polylysines and (iv) certain proteins or their derivatives such as gelatins. The cellulose derivatives are particularly preferred.

The contents of the different constitutive compounds of a pharmaceutical composition according to the invention are generally chosen such that the relative density of this composition in the stomach is less than 1.00.

Usually, a pharmaceutical composition according to the invention comprises from 5 to 70%, preferably from 10 to 60%, by weight of active principle, from 10 to 75%, preferably from 15 to 50%, by weight of at least one hydrophilic polymer and from 5 to 50%, preferably 10 to 40%, by weight of carbon dioxide generator agent, the percentages being expressed with respect to the total weight of the said composition.

A pharmaceutical composition for gastric residence according to the invention can be present in the form of gelatin capsules, granules or, preferably, tablets. The latter are floating tablets, that is to say they can float on the liquids of the stomach.

Such a pharmaceutical composition can be prepared by simple mixing of its components, followed by conversion to pharmaceutical form carried out in a conventional manner. Prior to the conversion to pharmaceutical form, in particular with a view to obtaining a floating tablet, the mixture comprising all or part of the constitutive components of the composition according to the invention can be granulated or agglomerated.

With a view to the preparation of a floating tablet according to the invention, it is possible to compress the mixture of constitutive components of the composition according to the invention. It is possible to add to the mixture to be compressed lubricating agents such as polyethylene glycols of molecular weight of between 1500 and 10,000, magnesium stearate or sodium stearyl fumarate, as well as conventional excipients such as flow agents or compression agents.

A pharmaceutical composition according to the invention, comprising a given benzamide, can be used for the treatment of illnesses already treated by the same benzamide in its conventional form. Thus, if the benzamide consists of amisulpride, sulpiride, one of their enantiomers, tiapride, or one of their salts, the pharmaceutical composition according to the invention can be used in the treatment of one of the pathologies mentioned further above, respectively, for each of these benzamides.

The examples which follow are intended to illustrate the present invention.

### EXAMPLE 1

### Floating Tablets Containing Tiapride Hydrochloride

The following compounds (% by mass) were mixed in a lemniscate mixer (Turbula®):

| | |
|---|---|
| tiapride hydrochloride | 37.0% |
| hydroxypropylmethylcellulose¹ | 30.0% |
| polyethylene glycol 6000 | 3.0% |
| anhydrous monosodium citrate | 16.8% |
| sodium bicarbonate | 13.2% |

¹hydroxypropylmethylcellulose 90 SH4000SR marketed by Shin-Etsu

The homogeneous mixture thus formed was compressed on an alternating compressing machine to obtain round, flat tablets of diameter 15 mm and comprising 300 mg of tiapride hydrochloride.

The dissolution of the tablets was tested according to the following method, using the paddle dissolution apparatus described in the European Pharmacopoeia:

the tablets were arranged in baskets revolving at 75 rpm and submerged in 1000 ml of 0.01 M hydrochloric acid at a temperature of 37±0.5° C. A sample of the medium, of volume 3 ml, was taken every hour up to four hours, then every two hours up to 12 hours. The quantity of tiapride was determined for each sample by U.V. spectrophotometry, in comparison with the absorbance of a standard solution containing 300 μg/ml of tiapride hydrochloride, in 0.01 M hydrochloric acid.

It was thus possible to determine the dissolution profile below:

| | hour | | | | | | | |
|---|---|---|---|---|---|---|---|---|
| | 1 | 2 | 3 | 4 | 6 | 8 | 10 | 12 |
| % dissolved | 12 | 18 | 19 | 27 | 35 | 42 | 44 | 51 |

### EXAMPLE 2

### Floating Tablets Containing Tiapride Hydrochloride

The following compounds (% by mass) were mixed in a lemniscate mixer (Turbula®):

| | |
|---|---|
| tiapride hydrochloride | 44.17% |
| hydroxypropylmethylcellulose¹ | 28.68% |
| magnesium stearate | 0.50% |
| sodium stearylfumarate | 2.87% |
| anhydrous monosodium citrate | 13.26% |
| anhydrous monosodium carbonate | 10.42% |
| Aérosil silica 200² | 0.10% |

¹hydroxypropylmethylcellulose 90 SH4000SR marketed by Shin-Etsu
²marketed by Degussa

The homogeneous mixture thus formed was compressed on an alternating compressing machine to obtain round, convex tablets of diameter 10 mm and comprising 200 mg of tiapride expressed as tiapride base.

The dissolution of the tablets was tested using the paddle dissolution apparatus described in the European Pharmacopoeia according to the following method:

the tablets were arranged in baskets of cylindrical shape, length 35 mm and diameter 19 mm, with perforations of diameter 5 mm. They were immersed in 1000 ml of 0.01 M hydrochloric acid at a temperature of 37±0.5° C. The dissolution medium was stirred by paddles revolving at 100 rpm. The medium was sampled every 15 minutes in a closed circuit by a peristaltic pump, and the quantity of tiapride determined by UV spectrophotometry in comparison with the absorbance of a standard solution containing 200 μg/ml of tiapride base, in 0.01 M hydrochloric acid.

It was thus possible to determine the dissolution profile below:

| | hour | | | | | | | | |
|---|---|---|---|---|---|---|---|---|---|
| | 1 | 2 | 3 | 4 | 6 | 8 | 10 | 12 | 14 |
| % dissolved | 24 | 33 | 42 | 49 | 63 | 76 | 86 | 93 | 98 |

To measure the flotation of the tablets, the tablet disintegration apparatus described in the European Pharmacopoeia was used under the following experimental conditions:

volume of water at 37° C.±1° C., 800 ml

stirring mechanism stopped in the low position

ends of the tubes open.

The tablets to be tested were introduced into 6 tubes. The tiapride hydrochloride tablets of Example 2 started to float at 2 min, and they continued to float for at least 120 min.

Pharmacokinetic Study of the Tablets of Example 2

The tablets of Example 2 were administered to 12 volunteers. Plasma samples were taken every 30 min from 0 to 3 h, every 2 h from 4 to 12 h, then at 16, 20, 24, 36 and 48 h after administration. The results are presented in the figure, which shows the mean plasma levels of tiapride base for the tablets of Example 2.

### EXAMPLE 3

### Floating Tablets Containing Amisulpride

The following compounds were mixed in a granulator/mixer type mixer:

| | |
|---|---|
| amisulpride | 39.4% |
| hydroxypropylmethylcellulose¹ | 29.8% |
| succinic acid | 15.9% |

¹hydroxypropylmethylcellulose 90 SH4000SR marketed by Shin-Etsu

The mixture was then granulated with 10% of water, and the granules dried in vacuo. After calibration, the granules were mixed with 13.7% sodium bicarbonate, then lubricated with 1% magnesium stearate and 0.2% Aerosil silica 200 (marketed by Degussa).

The homogeneous mixture thus formed was compressed on an alternating compressing machine to obtain round, convex tablets of diameter 10 mm and comprising 200 mg of amisulpride.

The dissolution method of Example 2 was used to determine the dissolution profile of the tablets thus prepared. The standard used was 200 μg/ml of amisulpride base. The following results were obtained:

| | hour | | | | | | | | |
|---|---|---|---|---|---|---|---|---|---|
| | 1 | 2 | 3 | 4 | 6 | 8 | 10 | 12 | 14 |
| % dissolved | 22 | 34 | 43 | 51 | 65 | 76 | 84 | 91 | 96 |

Their ability to float was tested by the method of Example 2. They started to float after 2 min, and they continued to float for at least 120 min.

### EXAMPLE 4

### Floating Tablets Containing (S)-(−)-amisulpride (D)-tartrate

Floating tablets of amisulpride were prepared according to the procedure described in Example 3. The tablets obtained comprised (% by mass) and were measured out to give a dose of 50 mg of (S)-(−)-amisulpride (D)-tartrate.

| | |
|---|---|
| (S)-(−)-amisulpride (D)-tartrate | 15.0% |
| 150 mesh lactose | 30.0% |
| hydroxypropylmethylcellulose¹ | 32.6% |
| magnesium stearate | 1.0% |
| tartaric acid | 10.0% |
| sodium bicarbonate | 11.2% |
| Aérosil silica 200² | 0.2% |

¹hydroxypropylmethylcellulose 90 SH4000SR marketed by Shin-Etsu
²marketed by Degussa

The dissolution method of Example 2 was used to determine the dissolution profile of the tablets thus prepared. The standard used was 50 μg/ml of amisulpride base. The following results were obtained:

| | hour | | | | | | | | |
|---|---|---|---|---|---|---|---|---|---|
| | 1 | 2 | 3 | 4 | 6 | 8 | 10 | 12 | 14 |
| % dissolved | 24 | 34 | 41 | 46 | 56 | 67 | 77 | 84 | 90 |

Their ability to float was tested by the method of Example 1. They started to float after 2 min, and they continued to float for at least 120 min.

### EXAMPLE 5

### Controlled-release Floating Tablets Containing Amisulpride

Floating amisulpride tablets were prepared according to the procedure described in Example 1.

The tablets obtained comprised (% by mass):

| | |
|---|---|
| amisulpride | 41.9% |
| hydroxypropylmethylcellulose¹ | 20.0% |
| sodium stearyl fumarate | 2.0% |
| magnesium stearate | 1.0% |
| anhydrous monosodium citrate | 20.0% |
| sodium bicarbonate | 15.0% |
| Aérosil silica 200² | 0.1% |

¹hydroxypropylmethylcellulose 90 SH4000SR marketed by Shin-Etsu
²marketed by Degussa

The dissolution method of Example 1 was used, modifying the times of taking samples to determine the dissolution profile of the tablets thus prepared. The following results were obtained:

| | hour | | | | | | |
|---|---|---|---|---|---|---|---|
| | 0.5 | 1 | 1.5 | 2 | 2.5 | 3 | 4 |
| % dissolved | 40 | 35 | 63 | 70 | 73 | 83 | 100 |

1. Pharmaceutical composition for gastric residence, characterized in that it comprises:
(a) an active principle consisting of a benzamide or a benzamide salt,
(b) a carbon dioxide generator system, and
(c) a means allowing the partial retention of the carbon dioxide generated by the said carbon dioxide generator system.

2. Composition according to claim 1, characterized in that the benzamide is tiapride or one of its salts.

3. Composition according to one of claims 1 and 2, characterized in that the benzamide is tiapride hydrochloride.

4. Composition according to claim 1, characterized in that the benzamide is amisulpride, one of its salts, one of its enantiomers or a salt of one of its enantiomers such as (S)-(−)-amisulpride (D)-tartrate.

5. Composition according to claim 1, characterized in that the benzamide is sulpiride, one of its salts, one of its enantiomers or a salt of one of its enantiomers.

6. Composition according to one of claims 1 to 5, characterized in that the carbon dioxide generator system comprises at least one carbon dioxide generator agent and at least one acidic compound chosen from the group formed by the monocarboxylic acids, the polycarboxylic acids and the partial salts of polycarboxylic acids.

7. Composition according to claim 6, characterized in that the carbon dioxide generator agent is an alkali metal or alkaline earth metal carbonate, such as calcium carbonate, or an alkali metal bicarbonate, such as sodium bicarbonate.

8. Composition according to one of claims 6 and 7, characterized in that the acidic compound is tartaric acid, succinic acid, citric acid or one of their partial salts such as monosodium citrate.

9. Composition according to one of claims 1 to 8, characterized in that the said means allowing the partial retention of the carbon dioxide generated by the said carbon dioxide generator system consists of at least one hydrophilic polymer.

10. Composition according to claim 9, characterized in that the hydrophilic polymer is a cellulose derivative, in particular hydroxypropylcellulose, hydroxypropylmethylcellulose and their mixtures.

11. Composition according to one of claims 1 to 10, characterized in that it is present in the form of a floating tablet.
